(19) United States
(12) Patent Application Publication
(10) Pub. No.: US 2017/0038243 A1
(43) Pub. Date: Feb. 9, 2017

(54) WEIGHT CALCULATING INTELLINGENT WEARABLE DEVICE AND METHOD THEREOF

(71) Applicant: Sheng Yu Peng, Taoyuan (TW)

(72) Inventor: Sheng Yu Peng, Taoyuan (TW)

(21) Appl. No.: 14/819,472

(22) Filed: Aug. 6, 2015

(51) Int. Cl. G01G 3/13; A61B 5/103; G01G 19/44; A43B 3/00

(52) U.S. Cl. CPC G01G 3/13; A43B 3/0005; A61B 5/1036; G01G 19/44

(57) ABSTRACT

Provided is a weight calculating intelligent wearable device and method thereof. The device includes a carrier and a data recorder. At least a heel piezoelectric element, at least a sole of foot piezoelectric element, a central control module, a power converting module and an electricity storage module are installed on the carrier. The heel piezoelectric element and the sole of foot piezoelectric element generate pulse signals when a heel of a wearer exerts pressure on the heel piezoelectric element during a walking process. The central control module includes a processor and a wireless transmitting unit. The pulse signals are received by the processor, and characteristic vectors are obtained by an algorithm. The characteristic vectors are transmitted from the wireless transmitting to the data recorder. A weight value of the wearer is obtained based on the characteristic vectors computed in a remote database or local data recorder.

### BACKGROUND OF THE INVENTION

1. Field of the Invention

The present invention relates to the technical field of a weight calculating intelligent wearable device and method thereof, and relates in particular to a design that is able to self-generate power and that can carry out weight calculation.

2. The Prior Arts

People of the modern day are often busy with work, and the situations of having high blood glucose, high blood cholesterol and high blood pressure are more and more common. These are severely harmful to health, and have become health worries of modern people. The partial factor that causes high blood glucose, high blood cholesterol and high blood pressure is obesity. As such, good self-weight management is beneficial towards reducing the incidence of diseases.

The development of communications network, together with innovative and practical designs enabled the emergence of various wearable devices, such as smart watches, electronic sports bracelet and the like. Such electronic products enable wearers to measure or record various physiological data while they are inactive or while they are exercising. These data can then stored in the remote database via the internet, or, the collected data can be analyzed and compared, thus enabling the wearers to easily understand the statuses of their own health and also enabling the wearers to carry out self-health control with convenience. However, there are limitations in using smart watches and electronic sports bracelets to collect the above-mentioned data. For example, these are not able to measure or collect the weight values of the wearers.

Taiwan Patent Publication No. 1476371 discloses an instant-type body measurement system. The system includes a weight measuring device and a mobile electronic device. The weight measuring device employs a static measuring mode. In the static measuring mode, a first pressure sensing unit and a second pressure sensing unit sense the pressure of a foot of the wearer. The pressure is converted to a weight numerical signal by a central processing unit. The weight numerical signal is transmitted to the mobile electronic device, and the weight value is displayed on the mobile electronic device. However, at a short time period, the measurement data can be interfered with noise generated by the pressure sensing elements and ambient temperature or wetting, such that accuracy of weight measurement is reduced. Moreover, power of the system is supplied by a power supply unit. When the power runs out, the power supply unit must be charged or must be replaced immediately. Accordingly, for the sake of meeting the requirement of providing a wearable body weight measuring device with self-power supply and high accuracy using a statistical and dynamic measurement data method, it is necessary to provide a wearable body weight measuring system that can measure body weight at any walking or running process.

### SUMMARY OF THE INVENTION

The main objective of the present invention is to provide a weight calculating intelligent wearable device and method thereof that are able to self-generate power and carry out weight calculation.

The other objective of the present invention is that the weight calculating intelligent wearable device and method thereof are mainly for receiving a plurality of pulse signals from the plurality of piezoelectric elements. The plurality of characteristic vectors may be obtained by algorithm calculations of the pulse signals. The most accurate weight value of the wearer may be calculated by means of using the plurality of characteristic vectors as well as the cloud database.

In order to achieve the above-mentioned objectives, the present invention includes a carrier and a data recorder. The carrier includes at least one heel piezoelectric element, at least one sole of foot piezoelectric element, a central control module and a power converting module. The electricity storage module may be installed on the carrier, whereby the heel piezoelectric element may transmit a pulse signal when the heel of a wearer exerts pressure on the heel piezoelectric element during a walking process. The sole of foot piezoelectric element may transmit the pulse signal when the heel of the wearer exerts pressure on the sole of foot piezoelectric element during the walking process. The central control module may include a processor and a wireless transmitting unit. The processor may be electrically connected with the heel piezoelectric element, the sole of foot piezoelectric element and the wireless transmitting unit. The processor may receive a plurality of pulse signals from the heel piezoelectric element and the sole of foot piezoelectric element. A plurality of characteristic vectors may be obtained by the processor, and may be transmitted by means of the wireless transmitting element. The power converting module may be electrically connected to the heel piezoelectric element, the sole of foot piezoelectric element and the power storage module. The power converting module may receive the plurality of pulse signals from the heel piezoelectric element and the sole of foot piezoelectric element, and the electricity storage module may be charged after power conversion. The electricity storage module may provide the electricity required for the weight calculating intelligent wearable device. The data recorder may receive the plurality of characteristic vectors transmitted by the wireless transmitting unit. The data recorder may transmit the plurality of characteristic vectors to a remote database, in order to connect with the remote database, and to calculate the weight of the wearer.

In addition, the present invention also provides a method for weight calculation, and the method is able to calculate the weight of a wearer during the walking process. The steps of the method are as follows. The foot pressure exerted by the wearer during the walking process may be received by at least a heel piezoelectric element and at least a sole of foot piezoelectric element, and a plurality of pulse signals are transmitted to a processor. The plurality of pulse signals may be received by the processor, and a plurality of characteristic vectors may be obtained by means of an algorithm. The plurality of characteristic vectors received by the processor may be transmitted to a data recorder by means of a wireless transmitting unit, and a weight value of the wearer may be obtained based on the plurality of characteristic vectors of the data recorder that are computed by a remote database or local data recorder.

The present invention respectively generates the plurality of pulse signals at the corresponding positions of the heel and the sole of foot of the wearer, after receiving the foot pressure by means of the plurality of piezoelectric elements. Besides its function of enabling the weight of the wearer to be calculated by means of algorithmic calculations and comparisons, the plurality of pulse signals are also able to perform the charging of the electricity storage module by means of the power converting module, and as such, the weight calculating intelligent wearable device of the present invention is able to generate power by its sustainability.

### BRIEF DESCRIPTION OF THE DRAWINGS

The present invention can be understood in more detail by reading the subsequent detailed description in conjunction with the examples and references made to the accompanying drawings, wherein:

FIG. 1 is an exploded view of the present invention.

FIG. 2 is a block diagram showing the operation of the various components within the carrier of the present invention.

FIG. 3 is a flow chart showing the method of the weight calculating device of the present invention.

FIG. 4 is a waveform diagram showing a single pulse signal of the present invention.

FIG. 5 is a waveform diagram showing the plurality of pulse signals generated by the plurality of piezoelectric elements during the walking process of a 60 kg person

FIG. 6 is a waveform diagram showing the plurality of pulse signals generated by the plurality of piezoelectric elements during the walking process of a 90 kg person.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) depict only the preferred embodiments of the invention and shall not be considered as limitations to the scope of the present invention. Modifications of the shape of the present invention shall be considered within the spirit of the present invention.

It should be noted that the structures, proportions, sizes and the like of the drawings of the current specification are only for depicting the disclosures of the present invention, to enable easy reading and understanding by persons skilled in the art, and these are not meant to be conditions that limit the present invention. As such, the drawings are not technologically meaningful. Any modifications of the structure, proportion relationships or any adjustments of the sizes should fall within the scope that is covered by the technical content of the present invention, without affecting the effects produced by the present invention and without affecting the goals of the present invention.

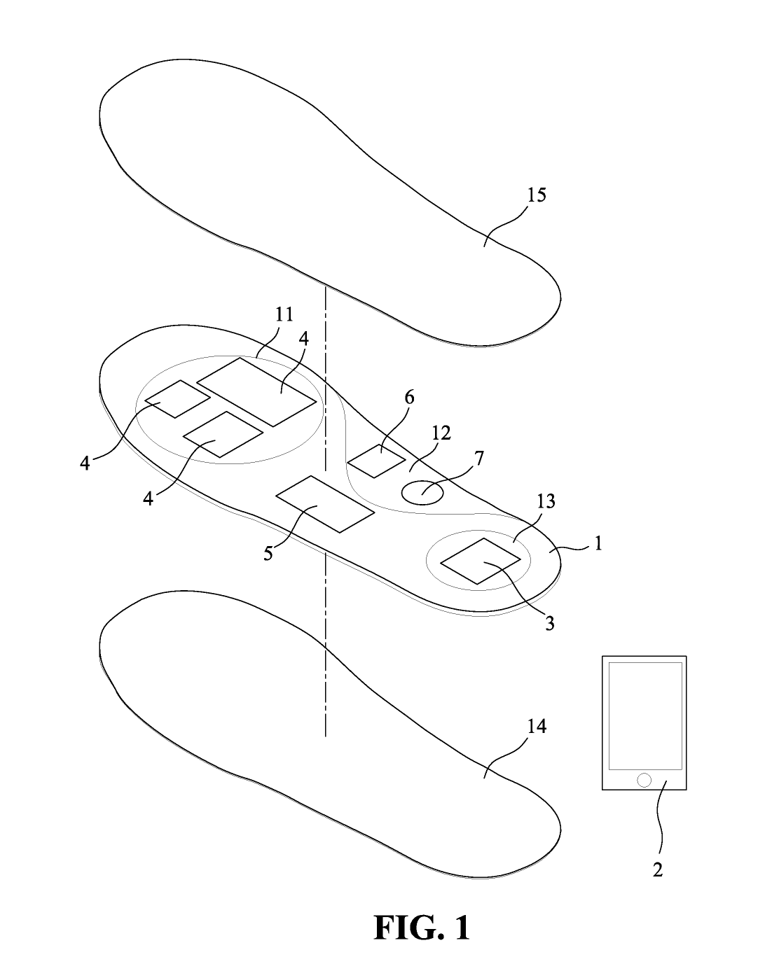

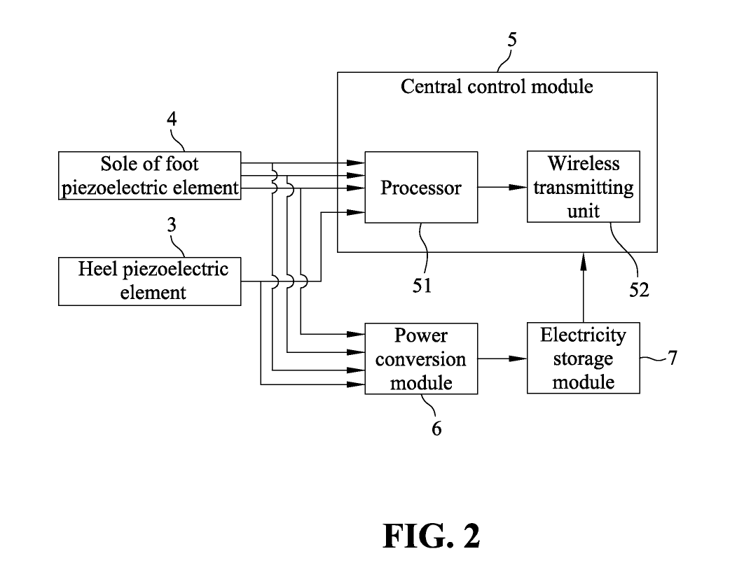

FIG. 1 is an exploded diagram of the present invention, and FIG. 2 is a block diagram showing some components of the present invention. The weight calculating intelligent wearable device includes a carrier 1 and a data recorder 2. The data recorder 2 may be a smartphone, a tablet PC and the like, which has the ability to receive the wireless communication signals and the ability to have comparisons with a remote database. The carrier 1 may be a soft plate, and at least one heel piezoelectric element 3, at least one sole of foot piezoelectric element 4, a central control module 5, a power converting module 6 and an electricity storage module 7 may be installed on the carrier 1. The carrier 1 may be a shoe pad, a shoe underlayer and an underlayer of a sock. The process of weight measurement may involve that the carrier 1 is in contact with the sole of the wearer.

The shape of the carrier 1 may correspond to the shape of the human foot, and may include a sole of foot region 11, a foot arch region 12 and a heel region 13. In accordance with a preferred embodiment of the present invention, there may be three sole of foot piezoelectric elements, which may be distributed in different positions of the sole of foot region 11. During the walking process of the wearer, the plurality of piezoelectric elements may generate a plurality of pulse signals as a result of receiving the foot pressure exerted by the wearer during the walking process. In accordance with another preferred embodiment of the present invention, a heel piezoelectric element exists in the heel region of the carrier 1, but is not limited to this. During the walking process of the wearer, the heel piezoelectric element 3 may be able to receive the pressure exerted by the heel and thus may transmit a pulse signal.

The central control module 5 may include a processor 51, a wire and a wireless transmitting unit 52. These may be integrated into a chip in accordance with the present day technology. The processor 51 may be electrically connected with the heel piezoelectric element 3, the sole of foot piezoelectric element 4 and the wireless transmitting unit 52. The processor 51 may receive the plurality of pulse signals from the heel piezoelectric element 3 and the sole of foot piezoelectric element 4. A plurality of characteristic vectors may be calculated by means of an algorithm, the plurality of characteristic vectors may be transmitted to the data recorder 2 by means of the wireless transmitting unit 52. The plurality characteristic vectors may include an output voltage, an output frequency as well as a peak voltage. The data recorder 2 may be connected to a remote database, and the data recorder takes into consideration the amplitude, pulse width and frequency characteristics of the pulse waves that are produced by persons having different weights who are walking or running. This enables the weight of the wearer to be calculated eventually, in accordance with the correction parameters of the piezoelectric elements and the comparison of the various parameters.

The power converting module 6 and the electricity storage module 7 may be installed within the foot arch region 12. Since such components have a large volume, the installation of such components in the foot arch region 12 may reduce the discomfort felt by wearers. The electricity storage module 7 may be a rechargeable battery, which may provide the necessary power to the various operating components. The power converting module 6 may be electrically connected to heel piezoelectric element 3, the sole of foot piezoelectric element 4 and the electricity storage module 7. The power converting module 6 may receive the pulse signals of the heel piezoelectric element 3 and the sole of foot piezoelectric element 4. The pulse signals may be converted to direct current (DC) signals. Furthermore, the electricity storage module 7 may be charged. Accordingly, the present invention is both convenient and practical, since it is able to self-generate power, and as such does not need an additional power source.

In addition, the top and bottom surfaces of the carrier 1 may further include a first waterproof protective layer 14 and a second waterproof protective layer 15. As such, the electronic elements installed within the carrier 1 may be protected. Also, the chance of the carrier 1 being out of order and being damaged may be lowered.

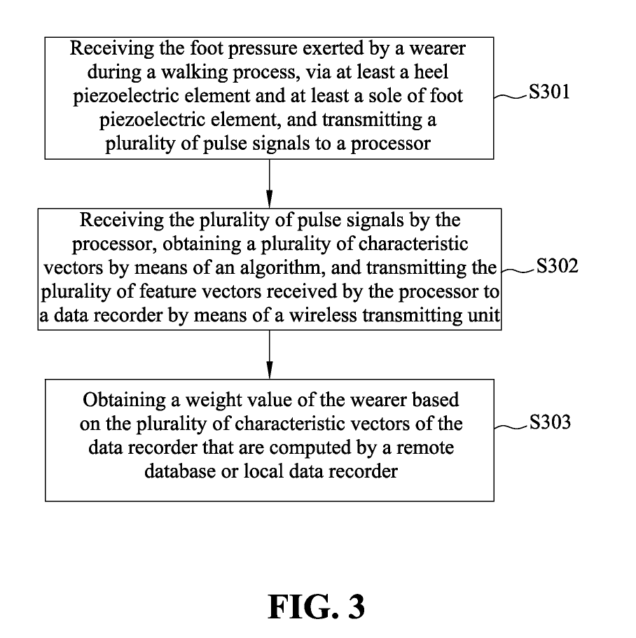

The following are descriptions of the method of the weight calculating intelligent wearable device of the present invention. Please refer also to FIG. 3, which shows the flow chart of the method of the present invention. The steps of the method are as follows.

The foot pressure exerted by the wearer during the walking process may be received by at least a heel piezoelectric element and at least a sole of foot piezoelectric element, and a plurality of pulse signals may be transmitted to a processor (Step S301).

The plurality of pulse signals may be received by the processor, a plurality of characteristic vectors may be obtained by means of an algorithm, and the plurality of characteristic vectors received by the processor may be transmitted to a data recorder, by means of a wireless transmitting unit (Step S302).

A weight value of the wearer may be obtained base on the plurality of characteristic vectors of the data recorder that are computed by a remote database (Step S303).

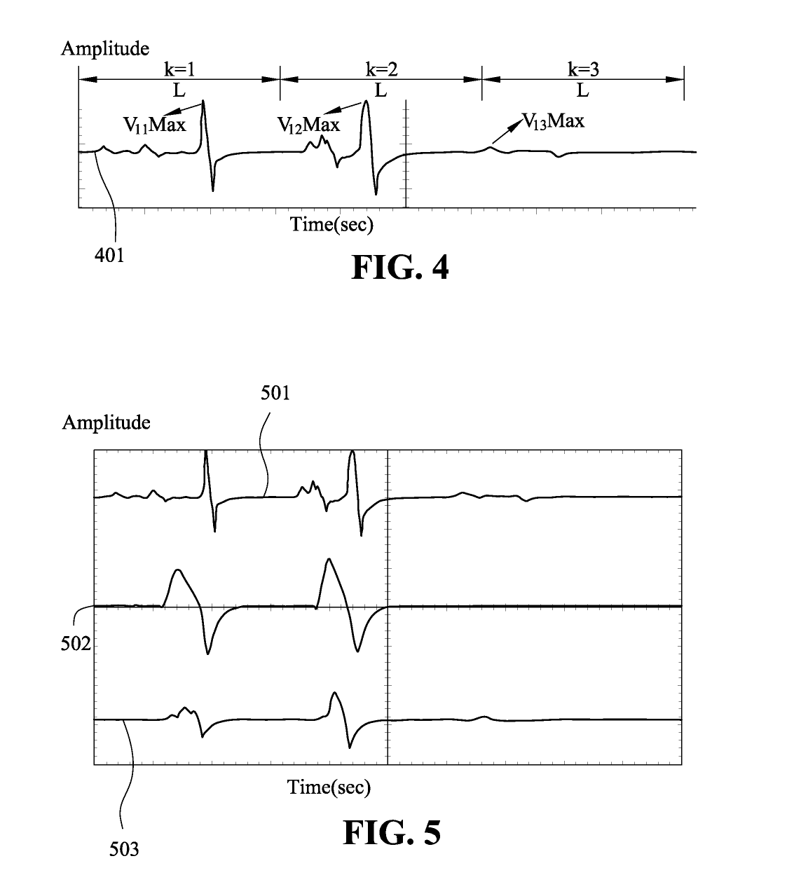

The output voltage may be calculated from the plurality of pulse signals obtained from an algorithm, by means of a processor. Referring to the waveform diagram of a pulse signal 401 as shown in FIG. 4, the pulse signal may be generated in response to the piezoelectric element (i.e., the sole of foot piezoelectric element) receiving the foot pressure exerted by the wearer. The algorithm may divide the pulse signal into kth window frame number, L may be the length of the window frame, and the formula for calculating the output voltage of the ith piezoelectric element is:

$${q}_{i} ( k ) = {ADJ}_{i} ( {T}_{k} , {\{ \frac{1}{L} {\sum }_{n = 1}^{L} {V}_{i} ( n + kL ) {V}_{i}^{*} ( n + kL ) \}}^{0.5} ) ,$$

where
L: length of window frame,
k: number of window frame,
V: output voltage of the ith piezoelectric element,
Tₖ: system environment temperature of the kth window frame,
V*: complex conjugate of the Vi signal,
ADJᵢ: correction parameter table of the piezoelectric element with regard to temperature and voltage, and
qᵢ(k): output voltage of the kth window frame in time after correcting the ith piezoelectric element.

The processor may process the output voltage together with the other information obtained such as the pulse signal, and as a result a characteristic vector may be defined. The characteristic vector Qi(k) is:

Qᵢ(k)=[qᵢ(k),V_{ikMAX},V_{iFreq}],

where

V_{ikMAX}: peak voltage of the ith piezoelectric element at the kth window frame in time,

V_{iFreq}: output frequency of the ith piezoelectric element,

qi(k): output voltage of the kth window frame in time after correcting the ith piezoelectric element.

In a preferred embodiment of the present invention, the plurality of characteristic vectors and a remote database may be connected by a data recorder, so as to compare the pre-established database parameters related to walking by the wearer. For example, data parameters such as the following may be considered in order to calculate the weight of a wearer: 1. the output voltage of the various piezoelectric elements; 2. position; 3. signal frequency; 4. peak voltage and the like.

In addition, the statistical data of the present invention may be based on a single-foot mode or 2-feet mode, and the plurality of characteristic vectors may be obtained based on the different modes, so as to enable the weight of the wearer to be calculated.

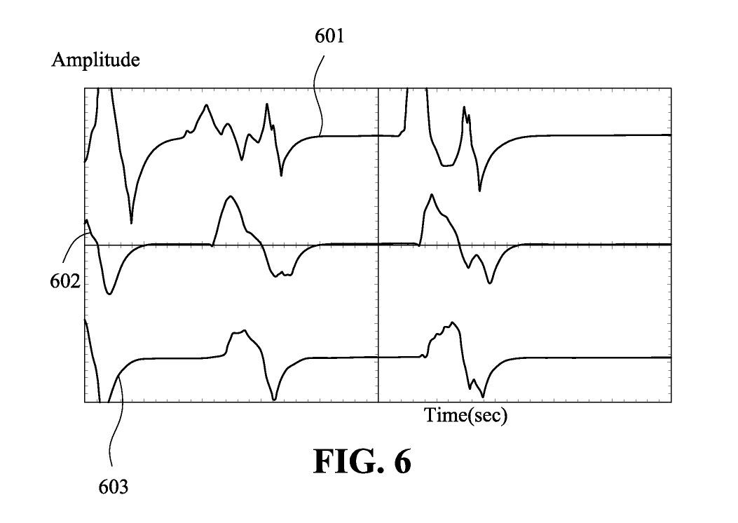

FIG. 5 is a waveform diagram showing the plurality of pulse signals 501, 502, 503 generated by the plurality of piezoelectric elements (i.e., the sole of foot piezoelectric element and two heel piezoelectric elements, respectively) during the walking process of a 60 kg person. FIG. 6 is a waveform diagram showing the plurality of pulse signals 601, 602, 603 generated by the plurality of piezoelectric elements (i.e., the sole of foot piezoelectric elements and two heel piezoelectric elements, respectively) during the walking process of a 90 kg person. It is clear from these FIGs. that different pulse signals may be generated by the walking of people having different weights. As such, when the database is established, a complete database may be established, by taking into consideration of and recording the various parameters such as the weight values of different people while they are walking or running. As a result, a more accurate weight value may be obtained later after comparisons are made using the data from the established database.

In view of the above, the weight calculating intelligent wearable device of the present invention has a design that is able to self-generate power and enable continuous weight measurements to be made. The weight measurements are mainly achieved by making use of the plurality of piezoelectric elements to generate the signals and the generation of electricity as a source. The correct weight of the wearer is calculated by means of the plurality of characteristic vectors and the remote database, and the weight measurements of the wearer may still be recorded while they are running or walking. Moreover, no additional power supply is required, since the weight calculating intelligent wearable device of the present invention may be self-recharged. In other words, it has an innovative and practical design. In addition, the design of (enabling) continuous weight measurements and self-generation of power, the present invention enables the majority of data obtained to be stored in the remote database, via the data recorder. Besides having effective weight control, various types of analyses can also be carried out, and thus achieving the objective of self-health management.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A weight calculating intelligent wearable device, comprising:
a carrier, wherein at least a heel piezoelectric element, at least a sole of foot piezoelectric element, a central control module, a power converting module and an electricity storage module are installed on the carrier,
wherein the heel piezoelectric element transmits a pulse signal when a heel of a wearer exerts pressure on the heel piezoelectric element during a walking process,
wherein the sole of foot piezoelectric element transmits the pulse signal when the heel of the wearer exerts pressure on the sole of foot piezoelectric element during the walking process,
wherein the central control module comprises a processor and a wireless transmitting unit, the processor is electrically connected with the heel piezoelectric element, the sole of foot piezoelectric element and the wireless transmitting unit, the processor receives a plurality of pulse signals from the heel piezoelectric element and the sole of foot piezoelectric element, a plurality of characteristic vectors are obtained by the processor, and are transmitted by means of the wireless transmitting element,
wherein the power converting module is electrically connected to the heel piezoelectric element, the sole of foot piezoelectric element and the power storage module, the power converting module receives the plurality of pulse signals from the heel piezoelectric element and the sole of foot piezoelectric element, and the electricity storage module is charged after power conversion;
wherein the electricity storage module provides electricity required for the weight calculating intelligent wearable device; and
a data recorder, receiving the plurality of characteristic vectors transmitted by the wireless transmitting unit.

2. The weight calculating intelligent wearable device of claim 1, wherein the plurality of characteristic vectors comprise an output voltage, an output frequency and a peak voltage.

3. The weight calculating intelligent wearable device of claim 1, wherein the carrier comprise one of a shoe pad, a shoe underlayer and an underlayer of a sock.

4. The weight calculating intelligent wearable device of claim 1, wherein the carrier is a soft plate, and a top surface and a bottom surface of the carrier comprise a first waterproof protective layer and a second waterproof protective layer, respectively.

5. The weight calculating intelligent wearable device of claim 1, wherein the sole of foot region piezoelectric element comprises a plurality of regions that correspond to a plurality of sole of foot regions.

6. A method for weight calculation, comprising the steps of:
receiving the foot pressure exerted by a wearer during a walking process, via at least a heel piezoelectric element and at least a sole of foot piezoelectric element, and transmitting a plurality of pulse signals to a processor;
receiving the plurality of pulse signals by the processor, and obtaining a plurality of characteristic vectors by means of an algorithm, transmitting the plurality of characteristic vectors received by the processor to a data recorder by means of a wireless transmitting unit; and
obtaining a weight value of the wearer based on the plurality of characteristic vectors of the data recorder that are computed by a remote database or local data recorder.

7. The method for weight calculation of claim 6, wherein the plurality of characteristic vectors comprise an output voltage, an output frequency and a peak voltage.

8. The method for weight calculation of claim 6, wherein the algorithm splits the plurality of pulse signals obtained by the ith piezoelectric element into k window frames, a length of each window frame is L, and the formula for calculating the output voltage of the ith piezoelectric element is:
$${q}_{i} ( k ) = {ADJ}_{i} ( {T}_{k} , {\{ \frac{1}{L} {\sum }_{n = 1}^{L} {V}_{i} ( n + kL ) {V}_{i}^{*} ( n + kL ) \}}^{0.5} ) ,$$
L: length of window frame;
k: number of window frame;
Vᵢ: output voltage of the ith piezoelectric element;
Tₖ: system environment temperature of the kth window frame;
Vi*: complex conjugate of the Vi signal;
ADJᵢ: correction parameter table of the piezoelectric element with regard to temperature and voltage;
qᵢ(k): output voltage of the kth window frame in time after correcting the ith piezoelectric element.
